(19) United States
(12) Patent Application Publication
(10) Pub. No.: US 2018/0264065 A1
(43) Pub. Date: Sep. 20, 2018

(54) SUPPLEMENTAL HEALTH BEVERAGE AND METHOD

(71) Applicant: Louis Parrott, Roosevelt, NY (US)

(72) Inventor: Louis Parrott, Roosevelt, NY (US)

(21) Appl. No.: 15/925,734

(22) Filed: Mar. 19, 2018

Related U.S. Application Data
(60) Provisional application No. 62/473,196, filed on Mar. 17, 2017.

(51) Int. Cl. A61K 36/81; A61K 36/41; A61K 36/254; A61K 36/16; A61K 36/03; A61K 36/481; A61K 36/258; A61K 36/74; A61K 36/424; A61K 36/31; A61K 36/484; A61K 36/58; A61K 36/77; A61K 36/23; A61K 31/07; A61K 31/51; A61K 31/525; A61K 31/455; A61K 31/197; A61K 31/4415; A61K 31/4188; A61K 31/519; A61K 31/714; A61K 31/375; A61K 31/593; A61K 31/355; A61K 31/122; A61K 31/145; A61K 31/198; A61K 31/20; A61K 33/42; A61K 33/06; A61K 33/30; A61K 33/04; A61K 33/34; A61K 33/24; A61K 33/00; A23L 2/52; A23L 2/60; A23L 2/02; A23L 2/56; A23L 2/54; A23L 2/68; A23L 2/44

(52) U.S. Cl. CPC A61K 36/81; A23V 2002/00; A61K 36/254; A61K 36/16; A61K 36/03; A61K 36/481; A61K 36/258; A61K 36/74; A61K 36/424; A61K 36/31; A61K 36/484; A61K 36/58; A61K 36/77; A61K 36/23; A61K 31/07; A61K 31/51; A61K 31/525; A61K 31/455; A61K 31/197; A61K 31/4415; A61K 31/4188; A61K 31/519; A61K 31/714; A61K 31/375; A61K 31/593; A61K 31/355; A61K 31/122; A61K 31/145; A61K 31/198; A61K 31/20; A61K 33/42; A61K 33/06; A61K 33/30; A61K 33/04; A61K 33/34; A61K 33/24; A61K 33/00; A23L 2/52; A23L 2/60; A23L 2/02; A23L 2/56; A23L 2/54; A23L 2/68; A23L 2/44; A61K 36/41

(57) ABSTRACT

A beverage for supplemental health; the beverage for supplemental health includes adaptogen herb tinctures, at least one flavoring agent, vitamins, minerals, at least one preservative and carbonated water. The beverage for supplemental health is useful for improving the overall health and wellness of an individual. The beverage for supplemental health is an admixture to promote health.

### CROSS-REFERENCE TO RELATED APPLICATION(S)

The present application is related to and claims priority to U.S. Provisional Patent Application No. 62/473,196 filed Mar. 17, 2017, which is incorporated by reference herein in its entirety.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

### TECHNICAL FIELD

The present invention relates generally to the field of body treating compositions of existing art and more specifically relates to an herbal remedy.

### RELATED ART

Many people are exposed to high levels of stress and fatigue at work and in other regular environments in life. High-stress exposure can put people at risk for numerous health issues such as depression, anxiety, weight gain and more. Excessive fatigue can lower the function of the immune system, leaving people susceptible to frequent and consistent illness. In the fast-paced world of today, the long-term effects of excessive stress and fatigue can lead to a myriad of mental and physical health issues. With people having so little time to focus on their health and wellness, it's no wonder there are millions of people suffering from preventable disease. A suitable solution is desired.

U.S. Pub. No. 2014/40057002 to Chen Xie relates to an anti-fatigue composition of plant material and preparation method, use and products thereof. The described anti-fatigue composition of plant material and preparation method, use and products thereof includes a composition of a plant material with anti-fatigue activity, its preparation method, usage and related products. The said composition is manufactured from the following plant materials: 20-80% from the genus Rhodiola; 10-60% Gynostemma; 10-60% Lycium. The said composition can be used to manufacture health product, food or anti-fatigue medicine which is able to significantly reduce the fatigue symptoms of those people suffering from fatigue, improve sleeping quality with no impact on normal body physiology. It is shown that the medicine derived from the present invention exerts its anti-fatigue effect through improving the functions of erythrocytes and thrombocytes to enhance the body's ability of supplying oxygen. The medicinal composition of the present invention is characterized by the small effective dose and good water solubility and thus suitable to be made into a variety of dosage forms for use.

### SUMMARY OF THE INVENTION

In view of the foregoing disadvantages inherent in the known herbal remedy art, the present disclosure provides a novel beverage for supplemental health. The general purpose of the present disclosure, which will be described subsequently in greater detail, is to provide an effective beverage for supplemental health.

A beverage for supplemental health is disclosed herein. The beverage for supplemental health includes at least one adaptogen herb tincture, at least one flavoring agent, at least one vitamin, at least one mineral, at least one preservative and carbonated water.

According to another embodiment, a method for making a supplemental health beverage is also disclosed herein. The method for making a supplemental health beverage includes mixing 200-5000 mg of adaptogen herb tinctures, mixing at least one flavoring agent, mixing at least one vitamin, mixing at least one mineral, mixing at least one preservative and combining mixture with carbonated water.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and methods of use for the present disclosure, a beverage for supplemental health, constructed and operative according to the teachings of the present disclosure.

FIG. 1 is a perspective view of the beverage for supplemental health during an ‘in-use’ condition, according to an embodiment of the disclosure.

FIG. 2 is a perspective view of the beverage for supplemental health of FIG. 1, according to an embodiment of the present disclosure.

FIG. 3 is a perspective view of the beverage for supplemental health of FIG. 1, according to an embodiment of the present disclosure.

FIG. 4 is a perspective view of the beverage for supplemental health of FIG. 1, according to an embodiment of the present disclosure.

FIG. 5 is a flow diagram illustrating a method for making a supplemental health beverage, according to an embodiment of the present disclosure.

The various embodiments of the present invention will hereinafter be described in conjunction with the appended drawings, wherein like designations denote like elements.

### DETAILED DESCRIPTION

As discussed above, embodiments of the present disclosure relate to an herbal remedy and more particularly to a beverage for supplemental health as used to improve the overall health and wellness of an individual.

Generally, the beverage for supplemental health may include a beverage, including adaptogen herb tinctures, vitamins, minerals and antioxidants to provide an overall boost to the body and mind's health and reaction to stress and fatigue. The beverage for supplemental health may further include a variety of flavors and carbonation to enhance the taste and enjoyability of the supplemental health beverage.

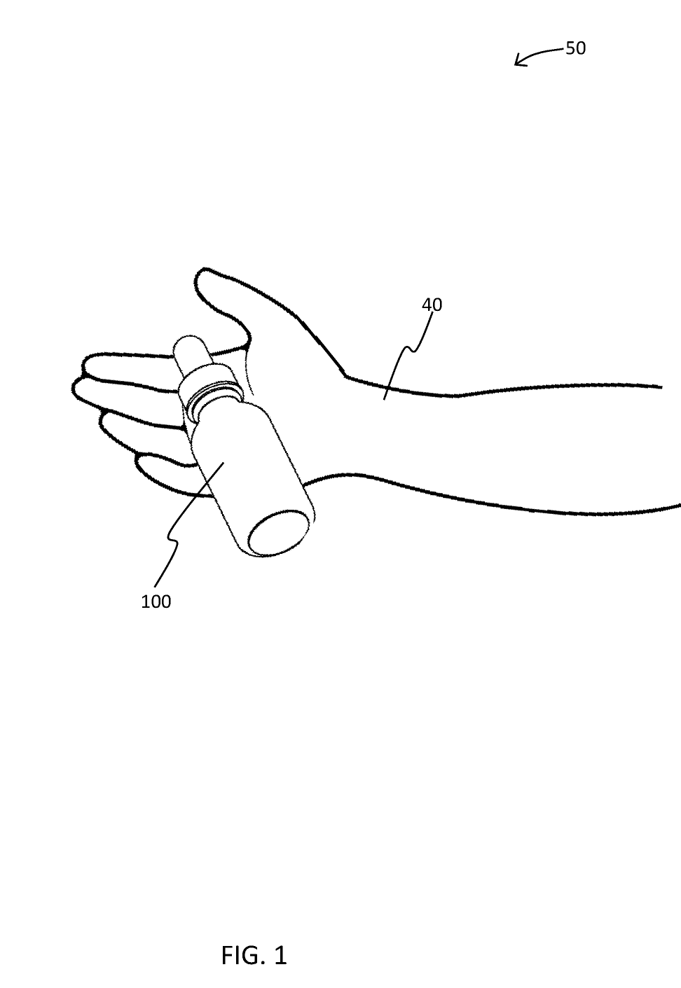

Referring now more specifically to the drawings by numerals of reference, there is shown in FIGS. 1-4, various views of a beverage for supplemental health 100.

FIG. 1 shows a beverage for supplemental health 100 during an ‘in-use’ condition 50, according to an embodiment of the present disclosure. Here, the beverage for supplemental health may be beneficial for use by a user 40 to improve the overall health and wellness of an individual. As illustrated, the beverage for supplemental health 100 may include adaptogen herb tinctures 110. Furthermore, the beverage for supplemental health 100 may include at least one flavoring agent 120 including, saccharides 310, at least one sweetener 320, and fruit juice 330. The beverage for supplemental health 100 may include vitamins 130, including at least one amino acid 400, and triglycerides 410. The beverage for supplemental health 100 may further include, minerals 140, at least one preservative 150, and carbonated water 160. Upon reading this specification it should be appreciated that, under appropriate circumstances, considering such issues as user preferences, design preference, structural requirements, marketing preferences, cost, available materials, technological advances, etc., other structural arrangements such as, for example, different flavoring agents, sweeteners, and packaging options, etc., may be sufficient.

According to one embodiment, the beverage for supplemental health 100 may be arranged as a kit 105. In particular, the beverage for supplemental health 100 may further include a set of instructions 107. The instructions 107 may detail functional relationships in relation to the structure of the beverage for supplemental health 100 such that the beverage for supplemental health 100 can be used, maintained, or the like, in a preferred manner.

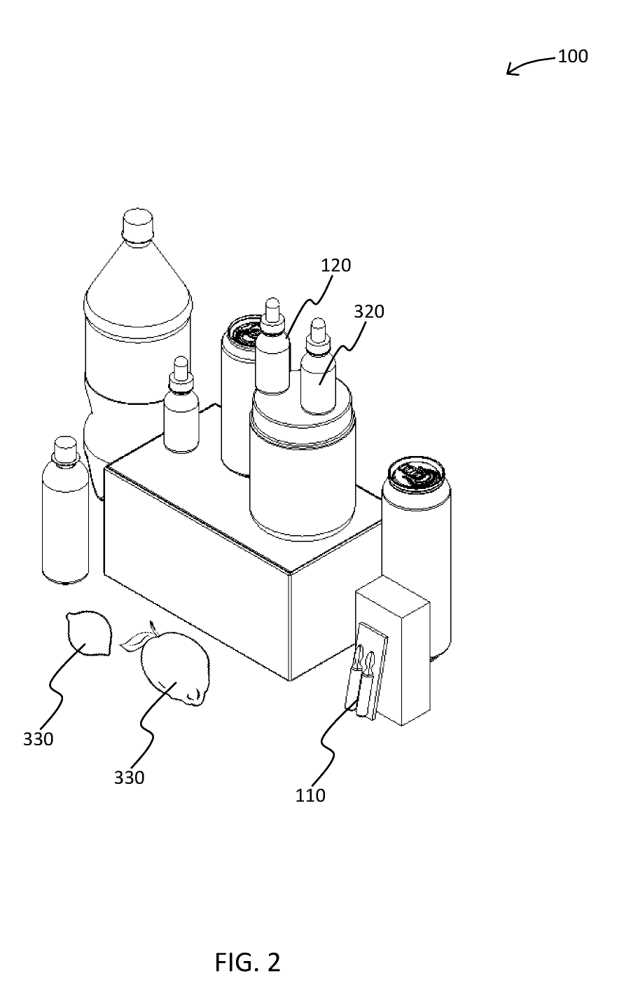

Referring now to FIG. 2 showing the beverage for supplemental health 100 of FIG. 1, according to an embodiment of the present disclosure. As above, the beverage for supplemental health 100 may include adaptogen herb tinctures 110. adaptogen herb tinctures 110 may include rhodiola rosea 200, eleuithero root 202, ginkgo leaf 204, sargassum 206, atragalus root 208, Asian ginseng root 210, coffee bean extract 212, jiaogulan 214, peruvian maca 216, American ginseng 218, Chinese liquorice root 220, catuaba 222, guarana 224, ashwagandha caffeine 226, purple carrot extract 228, parax ginseng 230, ginseng root extract 232 and ormus 234. Wherein the adaptogen herb tinctures 110 include 500-3000 mg of concentrated adaptogen herb tincture 110 per ounce of beverage.

At least one flavoring agent 120 may include citric acid 300, malic acid 302, and grade B maple syrup 304. Saccharides 310 may include 200 mg of at least one monosaccharide 311, at least one disaccharide 313, or at least one polysaccharide 316, per fluid ounce of beverage. At least one monosaccharide includes dextrose 312. At least one disaccharide includes isomaltulose 314, and sucralose 315. At least one polysaccharide includes maltodextrin 317, and waxy maize 318. At least one sweetener 320 includes acesulfame 322. Furthermore, fruit juice 330 includes a mixture of schisandra fruit juice 332, reishi fruit juice 334, jujube fruit juice 336, luohan guo fruit juice 338, acai berry juice 340, goji berry juice 342, lime juice 344, pear juice 346, and dragon fruit juice concentrate 348.

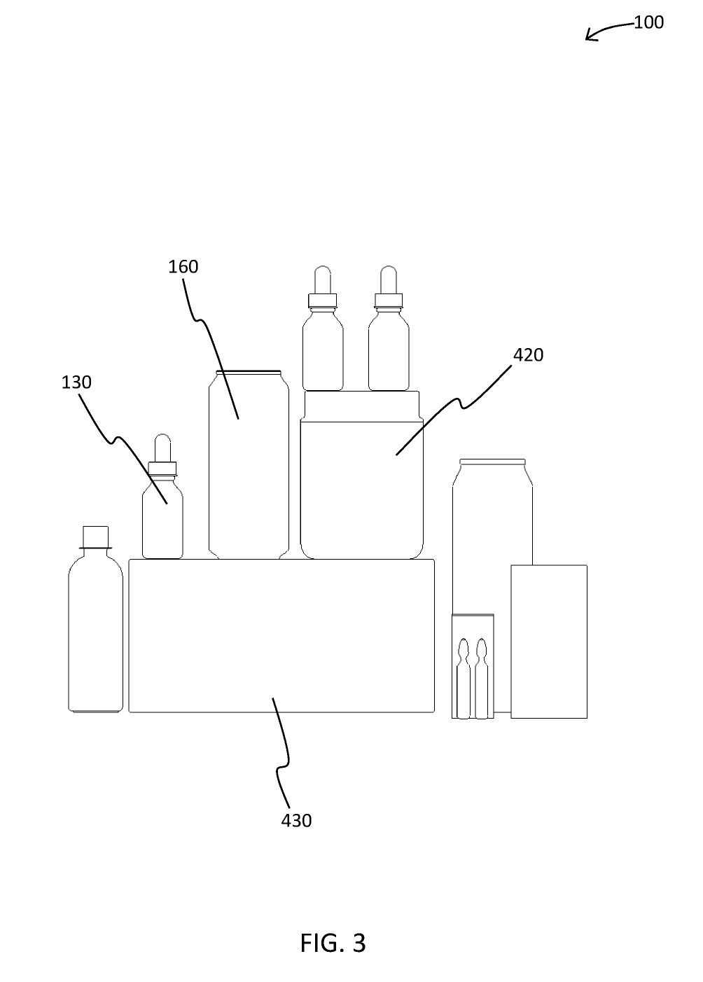

FIG. 3 is a view of the beverage for supplemental health 100 of FIG. 1, according to an embodiment of the present disclosure. According to this embodiment of the beverage for supplemental health 100, there are included vitamins 130, which include, at least 25% of the daily value of, vitamin A as beta-carotene 400, at least one B vitamin 410, vitamin C as ascorbic acid 402, vitamin D as cholecalciferol 404, vitamin E as d-alph tocopheryl acetate 406, and vitamin K as phytonadione per fluid ounce of beverage. Wherein at least one B vitamin 410 includes at least 25% of the daily value of thiamine 411, riboflavin 412, niacin 413, pantothenic acid 414, pyridoxine 415, biotin 416, folic acid 417, and cyanocobalamin 418 per fluid ounce of beverage. At least one amino acid 420 includes beta alanine 422, -taurine 424, and l-glutamine 426. Furthermore, wherein triglycerides 430, includes at least 25% of the daily value of medium chain triglycerides 432 per fluid ounce of beverage.

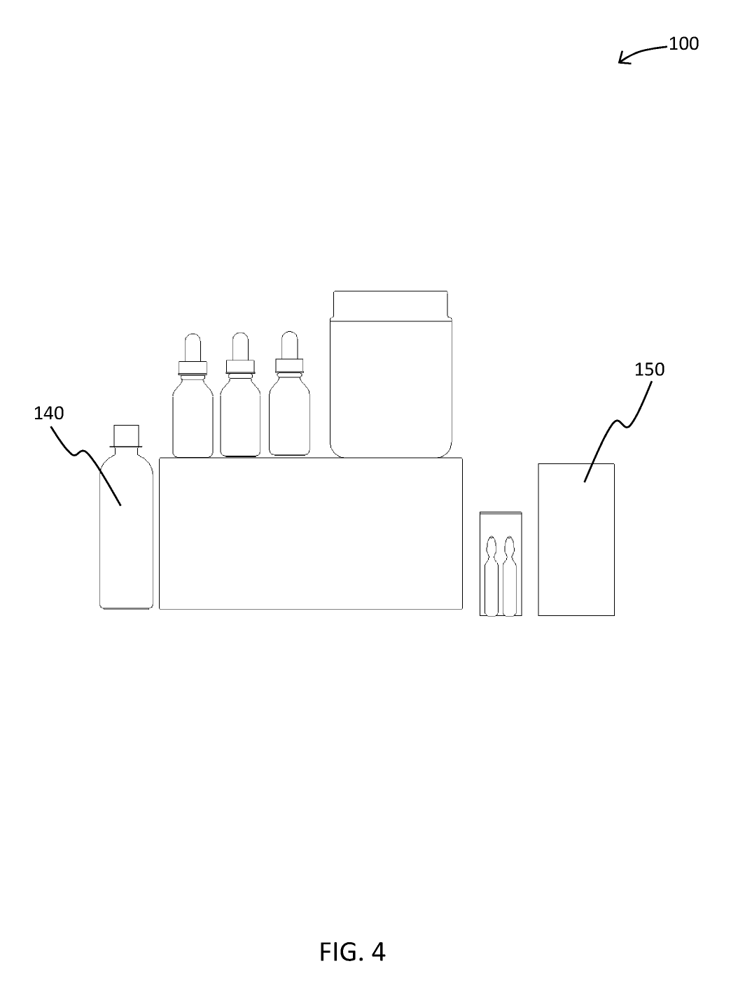

Referring now to FIG. 4, a perspective view of the beverage for supplemental health 100 of FIG. 1, according to an embodiment of the present disclosure. According to this embodiment of the beverage for supplemental health 100, there are included minerals 140. Minerals 140, may comprise at least 25% of the daily value of phosphorus 440, magnesium 441, zinc 442, selenium 443, copper 444, manganese 445, chromium 446 and potassium 447 per fluid ounce of beverage. Furthermore, at least one preservative 150 includes potassium sorbate 152.

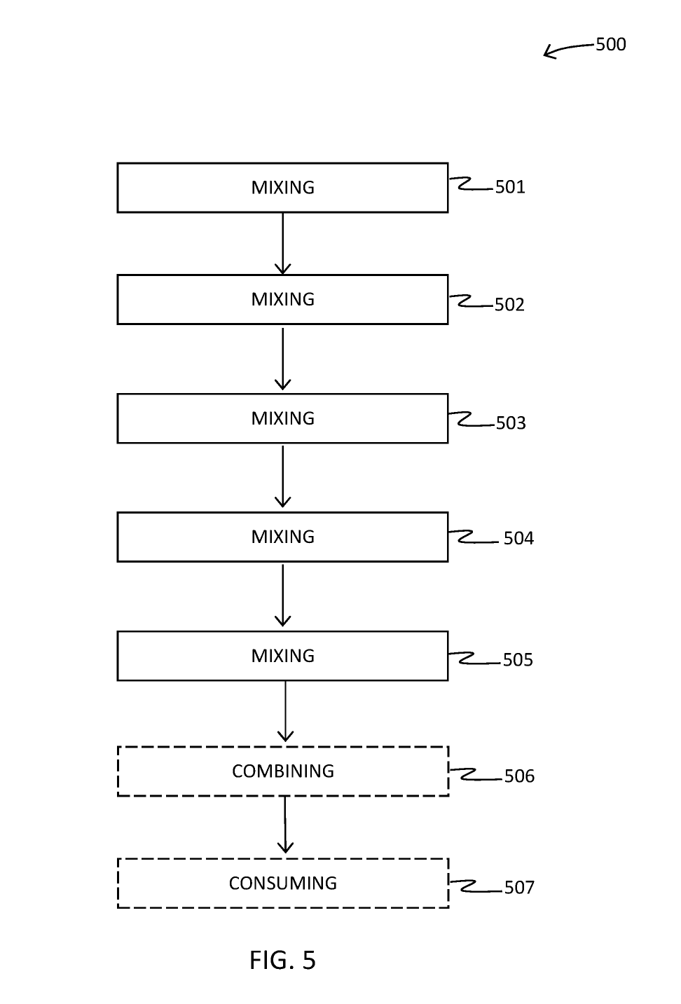

FIG. 5 is a flow diagram illustrating a method for making a supplemental health beverage 500, according to an embodiment of the present disclosure. In particular, the method for making a supplemental health beverage 500 may include one or more components or features of the beverage for supplemental health 100 as described above. As illustrated, the method for making a supplemental health beverage 500 may include the steps of: step one 501, mixing 300-5000 mg of adaptogen herb tinctures; step two 502, mixing at least one flavoring agent; step three 503, mixing vitamins; step four 504, mixing minerals; step five 505, mixing at least one preservative; step six 506, combining mixture with carbonated water; step seven 507, consuming a supplemental health beverage.

It should be noted that step 507 is an optional step and may not be implemented in all cases. Optional steps of method of use 500 are illustrated using dotted lines in FIG. 5 so as to distinguish them from the other steps of method of use 500. It should also be noted that the steps described in the method of use can be carried out in many different orders according to user preference. The use of “step of” should not be interpreted as “step for”, in the claims herein and is not intended to invoke the provisions of 35 U.S.C. § 112(f). It should also be noted that, under appropriate circumstances, considering such issues as design preference, user preferences, marketing preferences, cost, structural requirements, available materials, technological advances, etc., other methods for making a supplemental health beverage are taught herein.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A beverage for supplemental health, the beverage comprising:
an effective amount of adaptogen herb tinctures;
an effective amount of at least one flavoring agent including
saccharides,
at least one sweetener, and
fruit juice;
an effective amount of vitamins including,
at least one amino acid, and
triglycerides,
an effective amount of minerals;
an effective amount of at least one preservative; and
an effective amount of carbonated water; and
wherein said beverage for supplemental health comprises an admixture to promote health.

2. The beverage of claim 1, wherein the adaptogen herb tinctures further include rhodiola rosea, eleuithero root, ginkgo leaf, sargassum, atragalus root, Asian ginseng root, coffee bean extract, jiaogulan, Peruvian maca, American ginseng, Chinese liquorice root, catuaba, guarana, ashwagandha caffeine, purple carrot extract, parax ginseng, ginseng root extract and ormus.

3. The beverage of claim 2, wherein the adaptogen herb tinctures include 500-3000 mg of concentrated adaptogen herb tincture per ounce of beverage.

4. The beverage of claim 1, wherein the at least one flavoring agent includes citric acid, malic acid and grade B maple syrup.

5. The beverage of claim 1, wherein the saccharides include 200 milligrams of at least one monosaccharide, at least one disaccharide, or at least one polysaccharide per fluid ounce.

6. The beverage of claim 5, wherein the at least one monosaccharide includes dextrose.

7. The beverage of claim 5, wherein the at least one disaccharide includes isomaltulose and sucralose.

8. The beverage of claim 5, wherein the at least one polysaccharide includes maltodextrin and waxy maize.

9. The beverage of claim 1, wherein the at least one sweetener includes acesulfame.

10. The beverage of claim 1, wherein the wherein the fruit juice includes a mixture of schisandra fruit juice, reishi fruit juice, jujube fruit juice, luohan guo fruit juice, acai berry juice, goji berry juice, lime juice, pear juice concentrate and dragon fruit juice concentrate.

11. The beverage of claim 1, wherein the vitamins include at least 25% of the daily value of, vitamin A as beta-carotene, at least one vitamin B, vitamin C as ascorbic acid, vitamin D as cholecalciferol, vitamin E as d-alpha tocopheryl acetate, and vitamin K as phytonadione per fluid ounce.

12. The beverage of claim 11, wherein the at least one vitamin B includes, at least 25% of the daily value of thiamine, riboflavin, niacin, pantothenic acid, pyridoxine, biotin, folic acid, and cyanocobalamin per fluid ounce.

13. The beverage of claim 1, wherein the at least one amino acid includes beta alanine, -taurine, and l-glutamine.

14. The beverage of claim 1, wherein the plurality of triglycerides includes at least 25% of the daily value of medium chain triglycerides per fluid ounce.

15. The beverage of claim 1, wherein the minerals comprise at least 25% of the daily value of phosphorus, magnesium, zinc, selenium, copper, manganese, chromium and potassium per fluid ounce.

16. The beverage of claim 1, wherein the at least one preservative includes potassium sorbate.

17. A supplemental health beverage, the beverage comprising:
adaptogen herb tinctures;
at least one flavoring agent including
saccharides,
at least one sweetener, and
fruit juice;
vitamins including;
at least one amino acid, and
triglycerides;
minerals (140);
at least one preservative (150); and
carbonated water (160);
wherein the adaptogen herb tinctures further include rhodiola rosea, eleuithero root, ginkgo leaf, sargassum, atragalus root, Asian ginseng root, coffee bean extract, jiaogulan, Peruvian maca, American ginseng, Chinese liquorice root, catuaba guarana, ashwagandha caffeine, purple carrot extract, parax ginseng, ginseng root extract and ormus;
wherein the adaptogen herb tinctures include 500-3000 mg of concentrated adaptogen herb tincture per ounce of beverage;
wherein the at least one flavoring agent includes citric acid, malic acid and grade B maple syrup;
wherein the saccharides include at least one monosaccharide, at least one disaccharide, and at least one polysaccharide;
wherein the at least one monosaccharide includes dextrose;
wherein the at least one disaccharide includes isomaltulose and sucralose;
wherein the at least one polysaccharide includes maltodextrin and waxy maize;
wherein the at least one sweetener includes acesulfame;
wherein the wherein the fruit juice includes schisandra fruit juice, reishi fruit juice, jujube fruit juice, luohan guo fruit juice, acai berry juice, goji berry juice, lime juice, pear juice concentrate and dragon fruit juice concentrate;
wherein the vitamins include vitamin A as beta-carotene, at least one vitamin B, vitamin C as ascorbic acid, vitamin D as cholecalciferol, vitamin E as d-alpha tocopheryl acetate, and vitamin K as phytonadione;
wherein the at least one vitamin B includes, thiamine, riboflavin, niacin, pantothenic acid, pyridoxine, biotin, folic acid, and cyanocobalamin;
wherein the at least one amino acid includes beta alanine, taurine and l-glutamine;
wherein the plurality of triglycerides includes medium chain triglycerides;
wherein the minerals include phosphorus, magnesium, zinc, selenium, copper, manganese, chromium and potassium;
wherein the at least one preservative includes potassium sorbate; and
wherein said beverage for supplemental health comprises an admixture to promote health.

18. The beverage of claim 17, further comprising set of instructions; and
wherein the beverage is arranged as a kit wherein its components are packaged separately.

19. A method for making a supplemental health beverage, the method comprising the steps of:
mixing 300-5000 mg of adaptogen herb tinctures;
mixing at least one flavoring agent;
mixing vitamins;
mixing minerals;
mixing at least one preservative; and
combining mixture with carbonated water.

20. The method of claim 19, further comprising the step of
consuming a supplemental health beverage.
